(19) United States
(12) Patent Application Publication
(10) Pub. No.: US 2009/0281383 A1
(43) Pub. Date: Nov. 12, 2009

(54) APPARATUS AND METHOD FOR EXTERNAL FLUORESCENCE IMAGING OF INTERNAL REGIONS OF INTEREST IN A SMALL ANIMAL USING AN ENDOSCOPE FOR INTERNAL ILLUMINATION

(76) Inventors: Rao Papineni, Branford, CT (US); Gilbert Feke, Durham, CT (US)

Correspondence Address: Carestream Health, Inc., 150 Verona Street, Rochester, NY, 14608, US

(21) Appl. No.: 12/485,074

(22) Filed: Jun. 16, 2009

Related U.S. Application Data
(63) Continuation-in-part of application No. 11/221,530, filed on Sep. 8, 2005.
Application No. 11/221,530 is a continuation-in-part of application No. 12/381,599, filed on Mar. 13, 2009.
Application No. 12/381,599 is a continuation-in-part of application No. 12/475,623, filed on Jun. 1, 2009.
(60) Provisional application No. 61/075,033, filed on Jun. 24, 2008.

(51) Int. Cl. A61B 1/04

(52) U.S. Cl. 600/109

(57) ABSTRACT

An apparatus for capturing an optical molecular image of one or more internal regions of interest of a small animal having a cranio-caudal axis. The apparatus may include a support member for the animal in an immobilized state; an endoscope for insertion into and withdrawal from the body of the animal to allow the endoscope to be positioned selectively proximate the internal regions of interest within the body; a light source connected to the endoscope to provide illumination within the body at the regions of interest; a mechanism for rotating the animal about its cranio-caudal axis to enable image capture from different angles; and a device for capturing images of the one or more internal regions of interest from the different angles, due to light emitted from the one or more internal regions of interest through an external surface of the body in response to illumination from the light source. A corresponding method is disclosed.

### CROSS REFERENCE TO RELATED APPLICATIONS

Priority is claimed from commonly assigned, copending U.S. provisional patent application Serial No. 61/075,033 filed Jun. 24, 2008 by Papineni et al and entitled SMALL ANIMAL OPTICAL IMAGING ENDOSCOPES—GAVAGE SCOPES AND RECTAL SCOPES, the contents of which are incorporated by reference into the present specification.

This application is a continuation in part of the following commonly assigned, copending U.S. patent applications, the contents of each of which also are incorporated by reference into the present specification:

U.S. Ser. No. 11/221,530 filed Sep. 9, 2005 by Vizard et al., entitled APPARATUS AND METHOD FOR MULTI-MODAL IMAGING;

U.S. Ser. No. 12/381,599 filed Mar. 13, 2009 by Feke et al., entitled METHOD FOR REPRODUCING THE SPATIAL ORIENTATION OF AN IMMOBILIZED SUBJECT IN A MULTI-MODAL IMAGING SYSTEM; and

U.S. Ser. No. 12/475,623 filed Jun. 1, 2009 by Feke et al, entitled TORSIONAL SUPPORT APPARATUS AND METHOD FOR CRANIOCAUDAL ROTATION OF ANIMALS.

### FIELD OF THE INVENTION

The invention relates generally to the use of an endoscope for exciting optical or multimodal imaging probes, or both, located internally in a subject, such as a small animal, for molecular imaging. As used in this specification, the term “endoscope” includes not only the familiar illuminated, usually fiber-optic, flexible or rigid tubular instrument for visualizing the interior of a hollow organ or part (such as bladder, colon, stomach or esophagus) for diagnostic or therapeutic purposes that typically has one or more channels to enable passage of instruments (such as forceps or scissors); but also a flexible or rigid tubular, illuminated fiber optic instrument used simply to deliver illumination to such an organ or part of a body cavity, without including instruments or a means for examining the organ, part or cavity.

### BACKGROUND OF THE INVENTION

In known techniques for imaging of small subjects such as mice, the animals are treated with imaging probes that are targeted to locations in the body where a region of interest is located. A region of interest for example may be cancer cells, a tumor, or parts of various organs or tissue, all of which may be located deep within the subject. An often-encountered problem is that light applied from the exterior of the subject to excite the imaging probe dye to fluoresce may be of a wavelength that cannot readily pass through the tissue of the body, or is impeded by the endogenous tissue absorption, either of which may result in limited detection of the region of interest. In some cases even near infrared imaging probes are too deep to be excited by illumination applied externally and non-invasively. Another problem of known imaging techniques in which an imaging probe is excited by external illumination is that some probes can cause major portions of the body of the subject to fluoresce when the body is illuminated externally, in addition to the region of interest.

A variety of techniques are known for introducing illumination into the interior of a subject. U.S. Pat. No. 4,675,529 of Kushida discloses an endoscope useful for fluorescent spectral analysis, in which the probe both provides excitation illumination and collects light emitted within the subject. U.S. Pat. No. 4,898,175 of Noguchi discloses an apparatus for observing, from outside a human body, regions of interest within the body, by inserting an endoscope into the body via the mouth and then directing illumination from within the body toward an organ or region of interest. Light from the endoscope of Noguchi thus transilluminates the organ or region of interest so that light passes from the body to a camera outside the body that records the resultant image.

U.S. Pat. No. 5,501,225 of Wilson discloses an apparatus for detecting oxygen in tissue, in which a needle-guided probe is inserted, apparently transdermally, to a region of interest within the body of a subject, where it illuminates the region and causes it to produce phosphorescence emissions that can be detected outside the body by a camera. The probe is said to be moved from location to location to measure oxygen but the patent is unclear regarding whether the probe is moved along a single insertion and withdrawal track or is inserted again and again at spaced locations. U.S. Pat. No. 6,615,063 of Ntziachristos et al discloses time-lapse imaging to suppress auto fluorescence from body tissues not of interest. U.S. Patent Application Publication 2007/0016077 of Nakaoka et al and U.S. Patent Application Publication 2007/0087445 of Tearney et al each disclose endoscopes for fluorescent imaging in small animals such as mice.

U.S. Patent Application Publication 2007/0238957 of Yared discloses a combined X-ray and optical tomographic imaging system in which multiple emitters of excitation light and multiple detectors are used. U.S. Patent Application Publication 2007/0281322 of Jaffe et al describes bioanalytical instrumentation in which light sources in the form of a luminescent light pipe with relay optics irradiate molecules in a detection volume and direct fluorescence to one or more detectors.

U.S. Patent Application Publication 2008/0281322 of Ntziachristos describes using two forms of illumination to excite imaging probes in a subject, an epi-illumination light, which is reflected light; and a trans-illumination light, which is light that passes through the subject. In the method disclosed glass tubes are inserted into a dead mouse, the tubes are filled with a known dye or substance such as India ink. The image of the materials in the tubes are used to correct the images from both the epi-illumination and trans-illumination. Also Ntziachristos discloses using an endoscope to both epi-illuminate and trans-illuminate a human patient during an operation.

### SUMMARY OF THE INVENTION

The present inventors have recognized a need for a more efficient apparatus and method for exciting optical imaging probes internally located in a subject such as a small animal, for the purpose of molecular imaging. The inventors have discovered a technique for using an endoscope to provide focused internal illumination to excite such imaging probes in regions of interest, so that the probes emit light from within the subject that can be detected externally using a camera. The inventors have discovered an apparatus and method for sequentially imaging a region of interest with the subject rotated to various angles about its cranio-caudal axis, to improve accuracy of analysis. The region of interest may be imaged sequentially with the subject positioned at various angles about its cranio-caudal axis and at successive points along a path of movement of the endoscope within the subject, to provide a type of tomographic imaging. The use of focused internal illumination in accordance with this invention enables one to increase the depth penetration of imaging probes into the body of the subject and to image fluorescence from the probes from outside the body while using time-lapse techniques to simultaneously reduce autofluorescence.

An object of the present invention is to provide an apparatus and method for imaging internal regions of interest of a subject that have been treated with an imaging probe. Apparatus and method steps are provided for inserting an endoscope either orally or rectally into the subject, such as a mouse or small animal, placing the subject in an immobilized, anesthetized state on an object stage, capturing a set of molecular images, adjusting the position of the endoscope within the animal between each image capture in the series by moving or retracting the endoscope, and using a camera external of the subject for capturing the series of individual images of the regions of interest of the immobilized subject while keeping the camera at one location. In addition to or in place of adjusting the position of the endoscope, apparatus and method steps are provided for rotating the subject to various angles for additional images. The changes in the position of the endoscope can be monitored by low-energy X-ray imaging and used for the spatial determination and location of the images. The animal and the endoscope's inner light source may be turned or rotated together to different angles about the animal's cranio-caudal axis to obtain geometric localization of the target fluorescence or fluorescence resonance energy transfer. In another embodiment of the present invention a set of time domain images may be captured by illuminating the region of interest with the endoscope, delaying imaging until autofluorescence has decayed in other portions of subject's body, and then capturing fluorescence images from the region of interest. Time domain imaging permits in such a matter the elimination of autofluorescence of the endogenous tissue near the region of interest because of the time difference between the excitation of the probe by the excitation light source and the emission of light from the excited probes. In yet another embodiment, the animal may be anesthetized by injection; so that, the endoscope may be inserted by mouth without interference from a fixed inhalation unit for anesthesia.

An apparatus according to the invention is useful for capturing an optical molecular image of one or more internal regions of interest of a small animal having a cranio-caudal axis. The apparatus may include a support member for the animal in an immobilized state; an endoscope for insertion into and withdrawal from the body of the animal to allow the endoscope to be positioned selectively proximate such internal regions of interest within the body; a light source connected to the endoscope to provide illumination within the body at the regions of interest; means for rotating the animal about its cranio-caudal axis to enable image capture from different angles; and means for capturing images of one or more internal regions of interest from the different angles, due to light emitted from the one or more internal regions of interest through an external surface of the body in response to illumination from the light source.

A method according to the invention is useful for capturing an optical molecular image of one or more internal regions of interest of a small animal having a cranio-caudal axis. The method may include steps of immobilizing the animal on a support member; inserting an endoscope into the body of the animal to allow the endoscope to be positioned selectively proximate such internal regions of interest within the body; connecting a light source to the endoscope to provide illumination within the body at such regions of interest; rotating such an animal about its cranio-caudal axis to enable image capture from different angles; and capturing images of one or more internal regions of interest from the different angles, due to light emitted from one or more internal regions of interest through an external surface of the body in response to illumination from the light source.

### BRIEF DESCRIPTION OF THE DRAWINGS

FIG. 1 is a graphical representation of the logarithm of absorption by biological components of the body (molar extinction coefficient ε; absorbance α) versus excitation light (wavelength λ of excitation light) below 700 nm in the visible region of the spectrum. This plot is available from the Stowers Institute for Medical Research.

FIG. 2 shows a diagrammatic side view of a multimodal imaging system useful in the practice of the present invention.

FIG. 3 shows a diagrammatic front view of the imaging system of FIG. 2.

FIG. 4 shows a perspective view of the imaging system of FIGS. 2 and 3.

FIG. 5A shows a diagrammatic partial view of a mouse in a sample chamber, such as can be located on a sample object stage of the type illustrated for the imaging system of FIGS. 1 and 2.

FIG. 5B shows a single molecular image from a set of images of a region of interest within the mouse of FIG. 5A.

FIG. 6A shows a diagrammatic partial view of a mouse in a rotatable, translatable sample chamber in which an endoscope has been inserted into the mouse via its mouth, with the endoscope passing though an inhalation mask for anesthesia.

FIG. 6B shows a single molecular image from a set of images of regions of interest within the mouse of FIG. 6A.

FIG. 6C shows a diagrammatic partial view of a mouse in a rotatable, translatable sample chamber in which an endoscope has been inserted into the mouse via its mouth, but without passing through an inhalation mask, the mouse having been anesthetized by injection.

FIG. 6D shows a single molecular image from a set of images of regions of interest within the mouse of FIG. 6C.

FIG. 7A shows a diagrammatic partial view of a mouse in a sample chamber in which an endoscope has been inserted into the mouse via its rectum, with anesthesia passing though an inhalation mask to the nose and mouth of the mouse.

FIG. 7B shows a single molecular image from a set images of regions of interest within the mouse of FIG. 7A.

FIG. 7C shows a diagrammatic partial view of a mouse in a sample chamber in which an endoscope has been inserted into the mouse via its rectum, the mouse having been anesthetized by injection.

FIG. 7D shows a single molecular image from a set of images of regions of interest within the mouse of FIG. 7C.

FIG. 8 shows a work flow diagram in accordance with a method of the present invention.

### DETAILED DESCRIPTION OF THE INVENTION

The invention will be described in detail with particular reference to certain embodiments thereof, but it will be understood that variations and modifications can be effected within the spirit and scope of the invention. The following is a detailed description of various embodiments of the invention, reference being made to the drawings in which the same reference numerals identify the same elements of structure in each of the several figures.

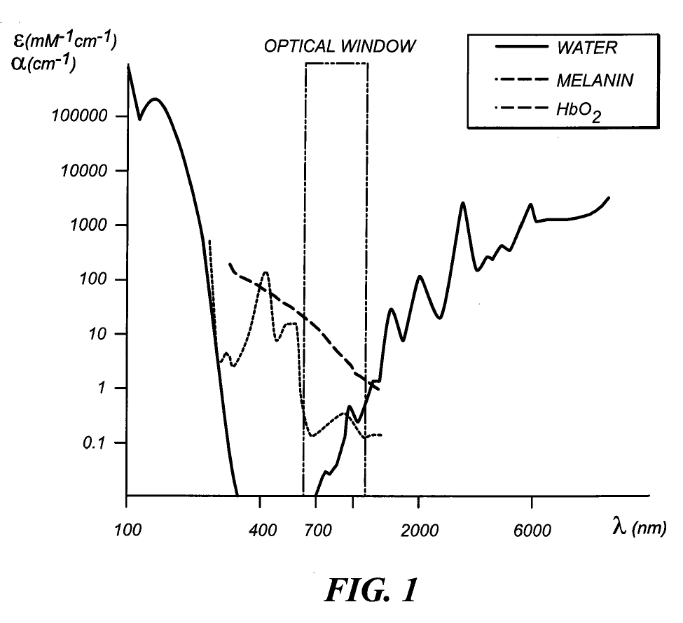

The present inventors have recognized a need for a more efficient apparatus and method for exciting optical imaging probes internally located in regions of interest of a subject such as a small animal for the purpose of molecular imaging. This invention provides a method to enhance and maximize the depth of tissue penetration of light from an excitation source. As shown in the graphical representation of FIG. 1, endogenous chromophores like hemoglobin (oxy-hemoglobin) and melanin absorb light and interfere by autofluorescence below 700 nm in the visible region of the spectrum. One embodiment of the method of the present invention enables one to avoid to a considerable extent this interfering autofluorescence and thus to attain high signal-to-background ratio. This is accomplished in accordance with the present invention since it permits one to increase the depth of penetration of excitation light and simultaneously to reduce autofluorescence by capturing a set of time domain images. Time domain imaging permits the elimination of autofluorescence of the endogenous bio tissue near the region of interest, because of the time difference between the excitation of the optical imaging probe by the excitation light source and the emission of light from the excited optical imaging probes. That is, excitation light causes fluorescence of both the imaging probe in the region of interest and the endogenous chromophores in other portions of the animal's body. The autofluorescence of the chromophores decays rapidly, in a fraction of a second typically; and following that decay, fluorescence from the region of interest may be captured without interference from autofluorescence.

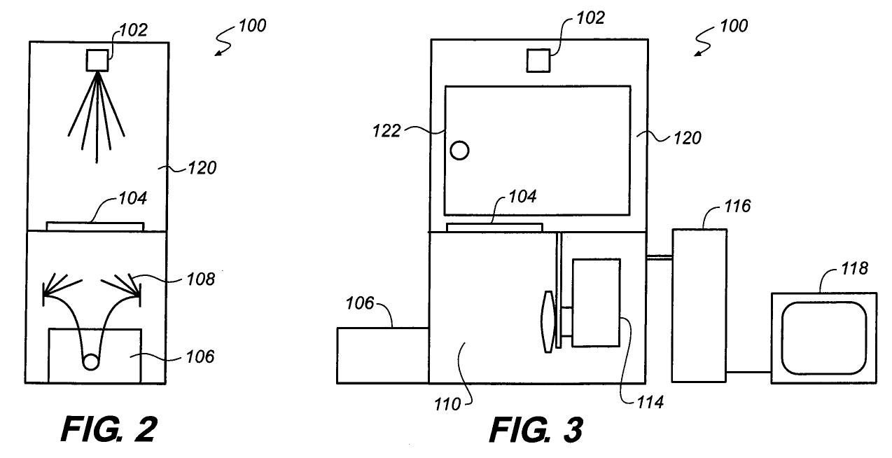

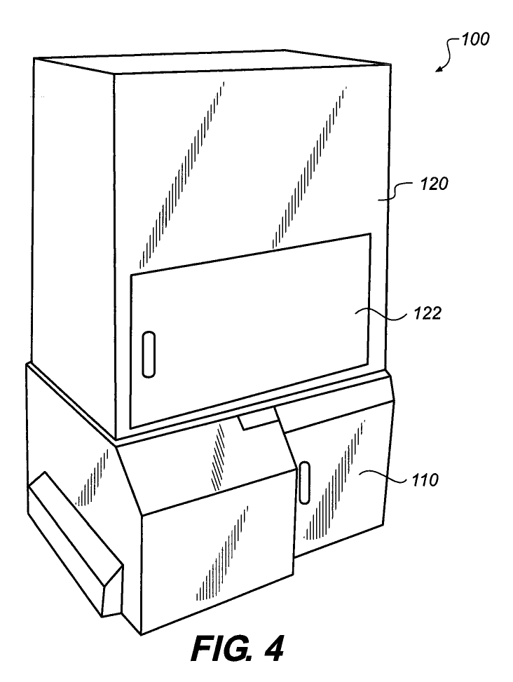

A multimodal imaging system 100 of a type useful for practice of the present invention is now described with reference to FIGS. 2, 3, and 4. Imaging modes useful for the apparatus and method of the present invention include x-ray imaging, dark-field imaging (fluorescence imaging) and radioactive isotope imaging. Images acquired in these modes can be merged or superimposed in various combinations for analysis. For example, an x-ray image of the subject can be merged with a near infrared fluorescence image of the subject captured in the same position, to provide a new image for analysis.

The imaging system 100 may be a multimodal type of imaging system such as a KODAK In-Vivo Imaging System FX Pro, which is of the general type described in the previously mentioned application of Vizard et al. Imaging system 100, in addition to an x-ray source 102, may include a programmable multispectral light source 106 capable of providing epi-illumination, fiber optics 108, an optical compartment 110, a means for capturing images such as a lens and camera system 114, and a communication and computer control system 116 with a display device 118, for example, a computer monitor.

A sample object stage 104 is disposed within a sample environment chamber 120, which allows access to the object being imaged. Sample environment chamber 120 may be light-tight and fitted with light-locked gas ports for environmental control. Such environmental control might be desirable for controlled x-ray imaging or for support of particular specimens. Imaging system 100 may include an access means or member 122 to provide convenient, safe and light-tight access to sample environment chamber 120. Access means are well known to those skilled in the art and can include a door, opening, labyrinth, and the like. Additionally, sample environment chamber 120 may be adapted to provide atmospheric control for sample maintenance or soft x-ray transmission (e.g., temperature/humidity/alternative gases and the like). Environmental control may be used to enable practical x-ray contrast below 8 KeV (air absorption) and to aid in life support for biological specimens.

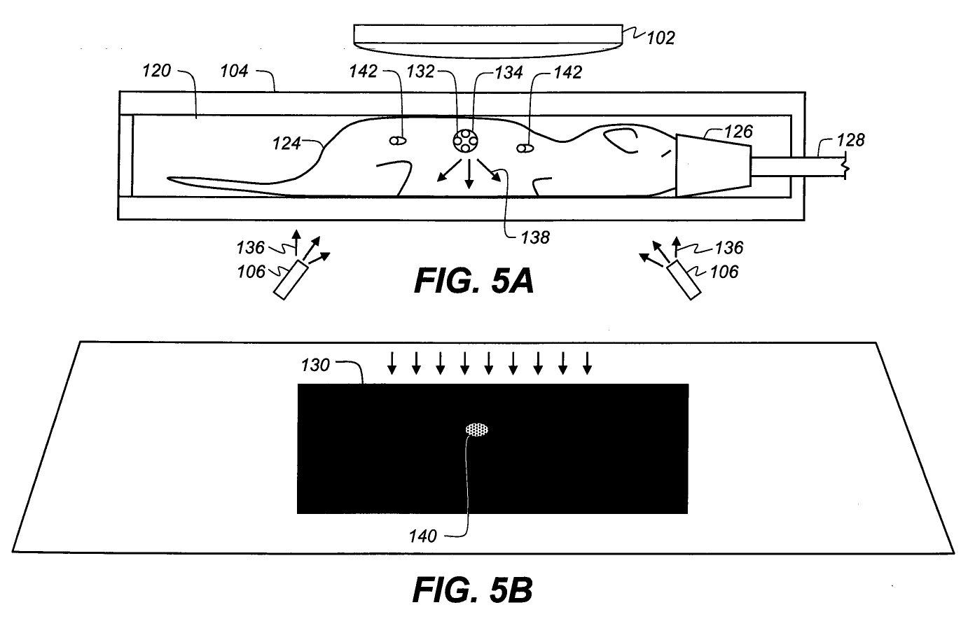

FIG. 5A shows a diagrammatic partial view of a subject such as a mouse 124 placed on sample object stage 104 of imaging system 100, for the purpose of acquiring a set 130 of molecular images, such as shown in FIG. 5B, in the manner disclosed in the previously mentioned pair of applications of Feke et al. Such images may include at least one of x-ray mode and dark-field mode (fluorescence). Mouse 124 is administered immobilizing anesthesia via a respiratory inhalation mask or device 126 connected to an outside source via a tube 128 which enters the sample environment chamber 120 via light-locked gas ports. Prior to being placed on stage 104, mouse 124 has been administered an imaging probe 132 which has been taken in by the cells of a region of interest such as cells of interest of a tumor 134. A set 130 of molecular images of the subject mouse's tumor 134 is acquired via the imaging system 100 and the imaging probes 132. To acquire set 130 of molecular images, the imaging probe is excited by externally applied excitation light 136 with a wavelength between 390 nm and 650 nm, which causes the imaging probe to emit light 138 between 390 nm and 830 nm. The amount of emitted light 138 passing outward through the mouse's body and reaching lens and camera system 114 depends on the depth of the location in the subject mouse 124 of the cells of interest or tumor 134, the strength of the imaging probe 132 and the wavelength of light 136 required to excite the imaging probe 132. As illustrated in FIG. 5A, externally applied excitation light 136 is emitted from a programmable, multispectral, external light source 106 and must pass through the tissue of mouse 124 to excite imaging probe 132. Because of the wavelength of excitation light 136 most of the energy reaching imaging probe 132 typically is absorbed by the tissue of mouse 124, thus greatly reducing the amount of light 138 emitted from the imaging probe 132 and hence reaching the lens and camera system 114, resulting in a lower quality molecular image 140 of the cells of interest or tumor 134. Only a single molecular image 140 is illustrated for set 130. Depending on the amount of excitation light 136 reaching the imaging probes 132 and the number of probes actually absorbed by the cells of interest, an image may not be acquired of nearby cells or interest or smaller tumors 142. In addition, if the optical imaging probe is excited and emits at the same time autofluorescence emits from the bio tissue components, the autofluorescence can mask the fluorescences from the optical imaging probes.

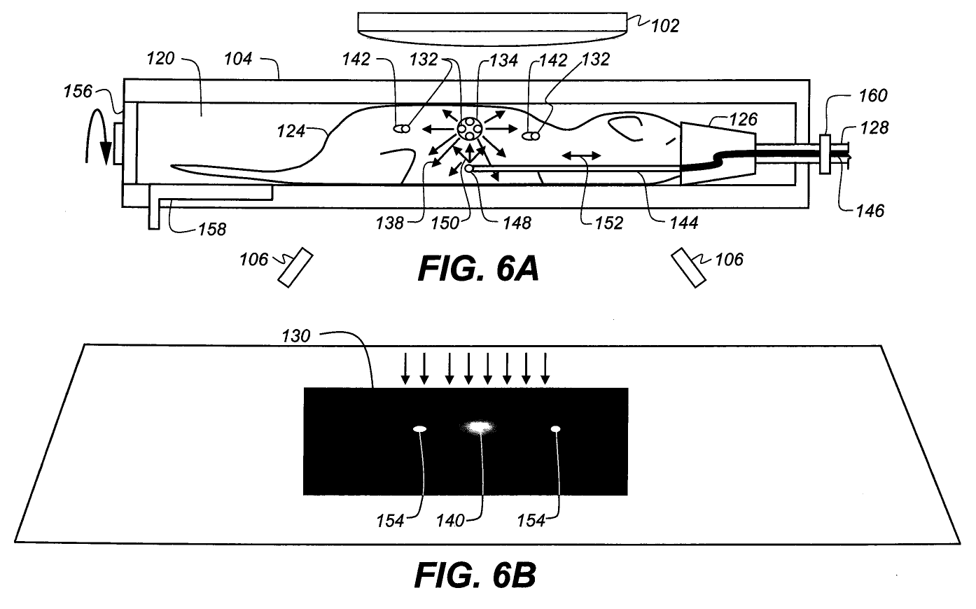

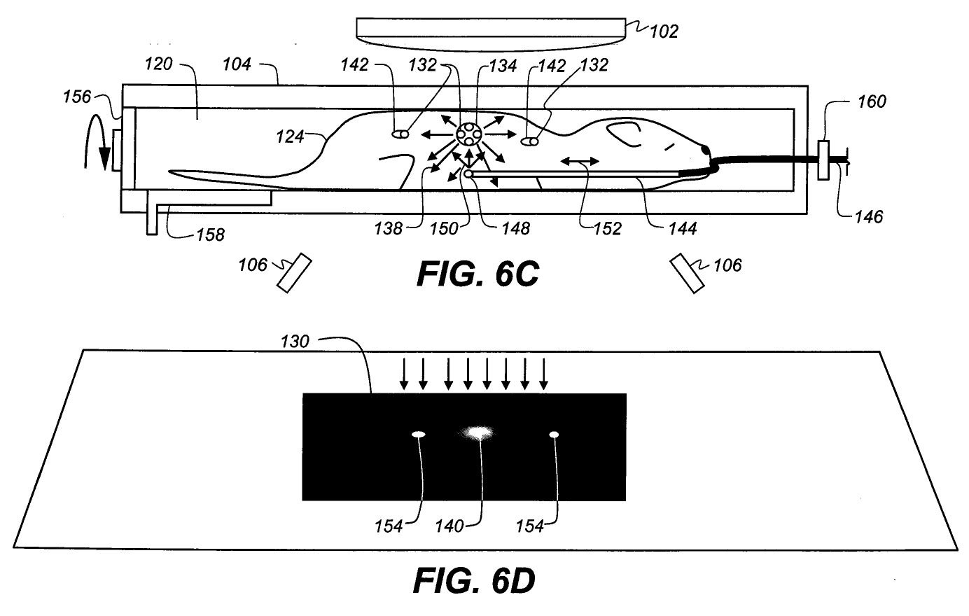

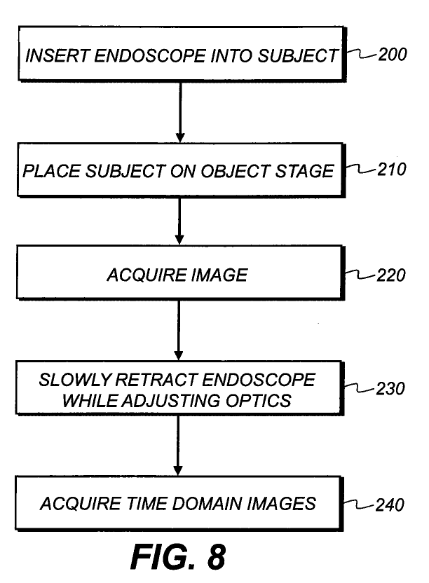

The apparatus and method of an embodiment of the present invention now are described with reference to FIGS. 6A to 6D and FIG. 8. An endoscope 144 is provided, having a connecting cable 146, which may include an optical fiber connected to light source 106 (connection not shown, for ease of illustration) or may include at its leading tip its own light source 148 such as an LED. Either source will make endoscope 144 capable of emitting a focused beam of excitation light 150 of a wavelength suitable for excitation of imaging probe 132. Endoscopes such as disclosed in the previously mentioned publications of Nakaoka et al and Tearney et al may be used. Light 150 may be adjusted in a manner familiar to those skilled in this technology to match the excitation wavelength of the particular imaging probe being used. Endoscope 144 is inserted gently, manually through the mouth of mouse 124, as shown at step 200 in the workflow of FIG. 8. At step 210 the mouse is placed on object stage 104. As shown in FIG. 6A, endoscope 144 may be threaded through and attached to inhalation mask 126. Alternatively, mouse 124 may be anesthetized by injection, so that the endoscope need not be attached to mask 126 or any similar fixed inhalation unit, as illustrated in FIG. 6C. By inserting endoscope 144 the excitation light source 148 may be positioned close to the imaging probes 132 in tumor 134. At step 220 an image 140 may be acquired. Then, in step 230, endoscope 144 may be further inserted into or retracted from mouse 124 in small increments, such as about 0.25 mm, minimum, as indicated by the arrow 152. Preferably, endoscope 144 is inserted manually to a maximum desired depth and then retracted. X-ray images may be used to confirm desired new positions of endoscope 144. Such incremental, gradual adjustments of the optics (that is, the position of endoscope 144) during retraction provide additional spatial resolution of fine targets that otherwise may not be imaged. For instance, as illustrated in FIGS. 6B and 6D, images 154 of the cells or regions of interest 142, that were not imaged during use of the external light source 106 of FIGS. 5A and 5B, are imaged in accordance with the invention due to internal illumination provided by repositioning endoscope 144. Also, as indicated in FIG. 6B, for example, molecular image 140 captured with internal illumination in accordance with the invention is much brighter than the same image in FIG. 5A captured with external illumination. Testing by the present inventors has shown that an increase in brightness of several-fold magnitude is achievable in accordance with the invention, thus enabling greatly improved imaging of internal organs and tumors using an external lens and camera system. The use of endoscope 144 in step 240 facilitates doing time domain imaging. During time domain imaging, excitation light 150 is turned on to excite the optical imaging probes and then turned off to allow any autofluorescence from surrounding bio tissue to decay, after which camera system 114 is operated to capture light emitted by the optical imaging probes in the cells or tumors of interest, thus greatly reducing the affect from the autofluorescence of the surrounding bio tissue.

Continuing with regard to FIGS. 6A to 6D, sample environment chamber 120 may be cylindrical in shape and may include means for rotating the mouse about its cranio-caudal axis during imaging, such as a rotational mechanism 156 for rotating the chamber; and an X-Y translation mechanism 158 for translating the chamber parallel to the cranio-caudal axis of the mouse, as disclosed in the previously mentioned applications of Feke et al. Mechanism 158 may be used to move chamber 120 incrementally to the left, as illustrated, so as to retract endoscope 144 for the purposes described in the preceding paragraph. Thus mechanism 158 and chamber 120 comprise a means for selectively withdrawing the endoscope. A conventional rotary joint 160 such as disclosed in U.S. Patent Application Publication 2006/0111613, shown schematically in FIGS. 6A and 6C, enables the inserted tip of endoscope 144 to remain stable within mouse 124 when chamber 120 and the mouse are rotated. That is, the portion of the endoscope within the mouse rotates with the mouse; so that, no relative rotation occurs between the two. Thus, mouse 124 may be rotated about its cranio-caudal axis to enable images 140, 154 to be captured by a fixed external camera 114 from various angles. Also, rotation of mouse 124 coupled with incremental movement of endoscope 144 during imaging, in the manner previously described, provide the ability to gather topographic information, similar to tomographic images.

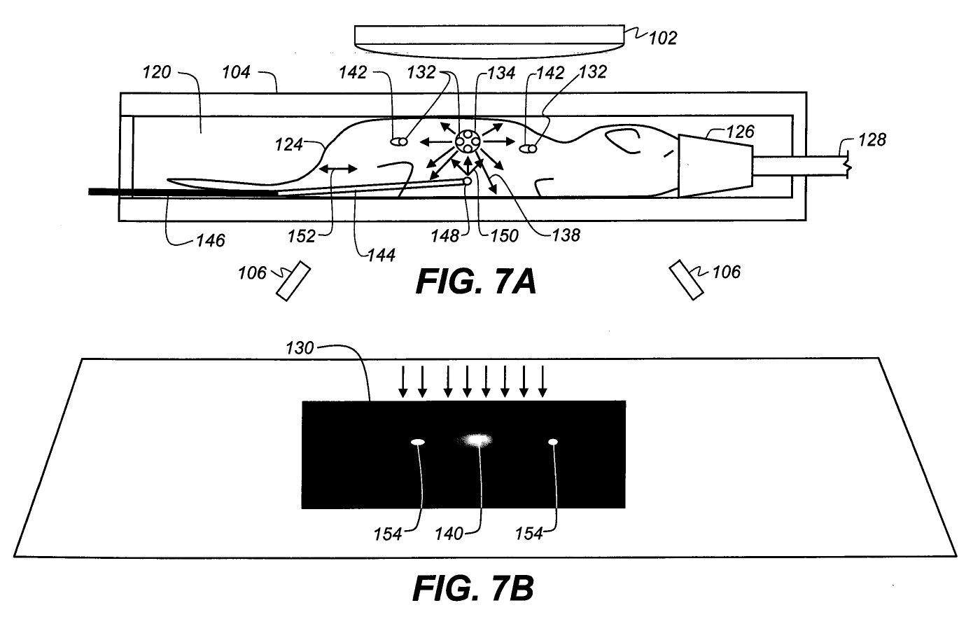

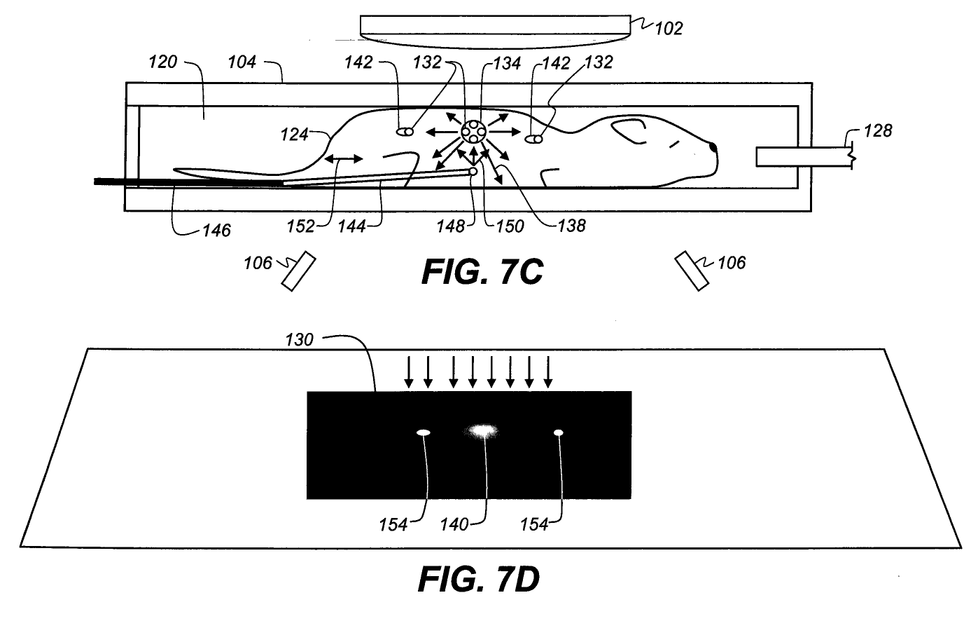

Another embodiment of the apparatus and method of the present invention is illustrated in FIGS. 7A to 7D. In this embodiment endoscope 144 is inserted via the rectum of mouse 124. Otherwise, this embodiment functions as described with regard to FIGS. 6A to 6D. Though not illustrated in FIGS. 7A to 7D, rotational mechanism 156, translation mechanism 158 and rotary joint 160 also could be included in this embodiment.

Rather than insertion via the mouth or rectum of mouse 124, endoscope 144 may be inserted into body cavities, spaces, or other regions of the mouse through fine syringe-needle equivalent entry points, such as disclosed in the previously mentioned patent of Wilson. In yet another embodiment, endoscope 144 may be made of a physiologically digestible optical fiber such as biological based, such as disclosed in U.S. Pat. No. 6,416,800 of Weber.

The invention has been described in detail with particular reference to certain preferred embodiments thereof, but it will be understood that variations and modifications can be effected within the spirit and scope of the invention.

### PARTS LIST

100 x-ray or radioisotopic imaging system
102 x-ray source
104 sample object stage or support member
106 external programmable multispectral light source
108 fiber optics
110 optical compartment
114 lens and camera system
116 communication and computer control system
118 display device
120 sample environment chamber
122 access means or member
124 subject animal, such as a mouse
126 respiratory inhalation mask or device
128 tube
130 set of molecular images of region of interest
132 imaging probe
134 cells of interest, such as a tumor
136 excitation light
138 emitted light
140 molecular image of set 130
142 nearby cells of interest or smaller tumors
144 endoscope
146 connecting cable
148 excitation light source
150 focused beam of excitation light from 144
152 arrow
154 molecular images of nearby regions of interest
156 rotational mechanism
158 X-Y translational mechanism
160 rotary joint for 144
200-240 steps of method

What is claimed is:

1. An apparatus for capturing an optical molecular image of one or more internal regions of interest of a small animal having a cranio-caudal axis, the apparatus comprising:
a support member for such an animal in an immobilized state;
an endoscope for insertion into and withdrawal from a body of such an animal to allow the endoscope to be positioned selectively proximate such internal regions of interest within such a body;
a light source connected to the endoscope to provide illumination within such a body at such regions of interest;
means for rotating such an animal about its cranio-caudal axis to enable image capture from different angles; and
means for capturing images of such one or more internal regions of interest from the different angles, due to light emitted from such one or more internal regions of interest through an external surface of such a body in response to illumination from the light source.

2. The apparatus according to claim 1 wherein the endoscope comprises a physiologically digestible optical fiber.

3. The apparatus according to claim 1, further comprising means for selectively withdrawing the endoscope from such an animal, whereby images may be captured at such one or more regions of interest at different positions of the endoscope.

4. The apparatus according to claim 3, wherein the endoscope is inserted and withdrawn via a mouth of such an animal.

5. The apparatus according to claim 3, wherein the endoscope is inserted and withdrawn via a rectum of such an animal.

6. The apparatus according to claim 3, wherein the means for selectively withdrawing moves the support member essentially parallel to a cranio-caudal axis of such an animal to withdraw the endoscope.

7. The apparatus according to claim 1, wherein the means for capturing captures images at times selected to allow decay of autofluorescence of portions of such an animal.

8. The apparatus according to claim 1, further comprising a rotary joint connected to the endoscope outside such an animal, to facilitate rotating such an animal without rotating the endoscope where it extends within such a body.

9. The apparatus according to claim 1, wherein the light source is programmable and multispectral.

10. The apparatus according to claim 1, further comprising an X-ray source for imaging a leading end of the endoscope as an aid for accurate positioning prior to imaging.

11. A method for capturing an optical molecular image of one or more internal regions of interest of a small animal having a cranio-caudal axis, the method comprising steps of:
immobilizing the animal on a support member;
inserting an endoscope into a body of the animal to allow the endoscope to be positioned selectively proximate such internal regions of interest within the body;
connecting a light source to the endoscope to provide illumination within the body at such regions of interest;
rotating such an animal about its cranio-caudal axis to enable image capture from different angles; and
capturing images of such one or more internal regions of interest from the different angles, due to light emitted from such one or more internal regions of interest through an external surface of the body in response to illumination from the light source.

12. The method according to claim 11, wherein the endoscope is inserted via a mouth of the animal.

13. The method according to claim 11, wherein the endoscope is inserted via a rectum of the animal.

14. The method according to claim 11, wherein images are captured at times selected to allow decay of autofluorescence of portions of the animal.

15. The method according to claim 11, further comprising a step of withdrawing the endoscope, wherein the images are captured at different positions of the endoscope during withdrawal of the endoscope.

16. The method according to claim 11, wherein the images are captured at different positions of the endoscope during insertion of the endoscope.

17. The method according to claim 11, further comprising a step of moving the support member essentially parallel to the cranio-caudal axis of the animal to withdraw the endoscope.

18. The method according to claim 11, wherein the light source is multispectral.

19. The method according to claim 11, further comprising a step of capturing an X-ray image of a leading end of the endoscope as an aid for accurate positioning prior to imaging.

20. An apparatus for capturing an optical molecular image of one or more internal regions of interest of a small animal, the apparatus comprising:
a support member for such an animal in an immobilized state;
an endoscope for insertion into and withdrawal from a body of such an animal to allow the endoscope to be positioned selectively proximate such internal regions of interest within such a body;
a light source connected to the endoscope to provide illumination within such a body at such regions of interest;
means for capturing an x-ray image of the animal with the endoscope inserted, for use in positioning the endoscope; and
means for capturing images of such one or more internal regions of interest due to light emitted from such one or more internal regions of interest through an external surface of such a body in response to illumination from the light source.

21. A method for capturing an optical molecular image of one or more internal regions of interest of a small animal, the method comprising steps of:
immobilizing the animal on a support member;
inserting an endoscope into a body of the animal to allow the endoscope to be positioned selectively proximate such internal regions of interest within the body;
connecting a light source to the endoscope to provide illumination within the body at such regions of interest;
capturing an x-ray image of the animal with the endoscope inserted, for use in positioning the endoscope; and
capturing images of such one or more internal regions of interest, due to light emitted from such one or more internal regions of interest through an external surface of the body in response to illumination from the light source.
